SEQUENCE LISTING (2 sequences)

SEQ ID NO: 1 - DNA, 26 bp, Artificial Sequence
  immunostimulatory oligonucleotide
ncncncncncncncncncncncncnc

SEQ ID NO: 2 - protein, 11 aa, Artificial Sequence
  polycationic oligopeptide
KLKLLLLLKLK

(19) United States
(12) Patent Application Publication
(10) Pub. No.: US 2011/0236489 A1
(43) Pub. Date: Sep. 29, 2011

(54) VACCINE ADJUVANT COMBINATIONS

(75) Inventors: Michele Pallaoro, Siena (IT); Derek O'Hagan, Winchester, MA (US); Rino Rappuoli, Castelnuovo Berardenga (IT)

(73) Assignee: NOVARTIS AG, Basel (CH)

(21) Appl. No.: 13/119,917

(22) PCT Filed: Sep. 18, 2009
(86) PCT No.: PCT/IB09/07111

§ 371 Date: Jun. 2, 2011

Related U.S. Application Data
(60) Provisional application No. 61/192,577, filed on Sep. 18, 2008.

(51) Int. Cl. A61K 9/14; A61K 39/145; A61P 37/04; A61P 31/16

(52) U.S. Cl. 424/489; 424/278.1; 424/209.1

(57) ABSTRACT

An immunological adjuvant comprises an oil-in-water emulsion, an immunostimulatory oligonucleotide and a polycationic polymer, wherein the oligonucleotide and the polymer ideally associate with each other to form a complex. The adjuvant can be combined with immunogens for preparing vaccines.

This application claims priority from U.S. provisional application 61/192,577 filed 18 Sep. 2008, the complete contents of which are hereby incorporated by reference.

### TECHNICAL FIELD

This invention is in the field of vaccine adjuvants and their combinations.

### BACKGROUND ART

Oil-in-water emulsions are known for use as vaccine adjuvants, and the FLUAD™ product includes the squalene-in-water ‘MF59’ adjuvant. It is an object of the invention to provide modified and improved emulsion adjuvants.

### DISCLOSURE OF THE INVENTION

The invention provides an immunological adjuvant comprising an oil-in-water emulsion, an immunostimulatory oligonucleotide and a polycationic polymer. The oligonucleotide and the polymer ideally associate with each other to form a complex.

The invention also provides a process for preparing an immunological adjuvant of the invention, comprising a step of mixing an oil-in-water emulsion with a complex of an immunostimulatory oligonucleotide and a polycationic polymer.

The invention also provides an immunogenic composition comprising (i) an adjuvant of the invention and (ii) an immunogen.

The invention also provides a process for preparing an immunogenic composition comprising a step of mixing (i) an adjuvant of the invention and (ii) an immunogen.

The immunogen, emulsion, oligonucleotide and polymer may be mixed in any order. For example, the invention provides a process for preparing an immunogenic composition of the invention, comprising a step of mixing (i) an oil-in-water emulsion and (ii) an immunogen; and then mixing the emulsion/immunogen mixture with an immunostimulatory oligonucleotide and a polycationic polymer.

The invention also provides a process for preparing an immunogenic composition of the invention, comprising a step of mixing (i) an immunostimulatory oligonucleotide and a polycationic polymer, typically in the form of a complex, and (ii) an immunogen; and then mixing the oligonucleotide/polymer/immunogen mixture with an oil-in-water emulsion.

The invention also provides a kit comprising: (i) a first container that contains an adjuvant of the invention; and (ii) a second container that contains an immunogen and/or a further adjuvant. The invention also provides a kit comprising: (i) a first container that contains an oil-in-water emulsion; and (ii) a second container that contains an immunostimulatory oligonucleotide and a polycationic polymer. One or both of the first and second containers may include an immunogen. Thus the contents of the two containers can be combined (e.g. at the point of use) to form an adjuvant or immunogenic composition of the invention. These kits may include a third container that contains an immunogen and/or a further adjuvant.

The invention also provides an immunological adjuvant comprising an oil-in-water emulsion and an adsorptive particulate adjuvant, wherein the average diameter of particles in the adsorptive particulate adjuvant and the average diameter of oil droplets in the emulsion are both less than 250 nm (e.g. ≦220 nm, ≦200 nm, ≦190 nm, ≦180 nm, ≦150 nm, ≦120 nm, ≦100 nm, etc.). If the particulate adjuvant has particles with a range of diameters then these diameters may not overlap with the oil droplet particles sizes (i.e. the largest oil droplets are smaller than the smallest adjuvant particles, or the largest adjuvant particles are smaller than the smallest oil droplets). In other embodiments, however, the size distributions may overlap. In some embodiments the average diameter of the adjuvant particles may be substantially the same as the average diameter of the oil droplets, or these two diameters may differ e.g. by at least 5%, 10%, 15%, 20%, 25%, etc.

The invention also provides an immunological adjuvant comprising an oil-in-water emulsion and an adsorptive particulate adjuvant, wherein the average diameter of particles in the adsorptive particulate adjuvant is greater than the average diameter of oil droplets in the emulsion. The adsorptive particulate adjuvant ideally does not comprise (i) an insoluble aluminium or calcium salt or (ii) polymeric microparticles, but rather is preferably (iii) a complex of an immunostimulatory oligonucleotide and a polycationic polymer. Mixing an oil-in-water emulsion with such complexes is shown herein to reduce the complexes' analysed mean diameter.

The invention also provides an immunogenic composition comprising (i) an immunological adjuvant comprising an oil-in-water emulsion and an adsorptive particulate adjuvant; and (ii) an immunogen. As described above, the adsorptive particulate adjuvant ideally does not comprise an insoluble aluminium or calcium salt or a polymeric microparticle but is preferably a complex of an immunostimulatory oligonucleotide and a polycationic polymer. Typically, the immunogen is at least partially adsorbed to the adsorptive particulate adjuvant.

The invention also provides an immunological adjuvant comprising an oil-in-water emulsion and an immunostimulatory oligonucleotide, wherein the immunostimulatory oligonucleotide includes at least one CpI motif (a dinucleotide sequence containing a cytosine linked to an inosine). The oligodeoxynucleotide may include more than one (e.g. 2, 3, 4, 5, 6 or more) CpI motif, and these may be directly repeated (e.g. comprising the sequence (CI)ₓ, where x is 2, 3, 4, 5, 6 or more) or separated from each other (e.g. comprising the sequence (CIN)ₓ, where x is 2, 3, 4, 5, 6 or more, and where each N independently represents one or more nucleotides). Cytosine residues in the oligonucleotide are ideally unmethylated. The invention also provides a process for preparing this immunological adjuvant, comprising a step of mixing an oil-in-water emulsion with a CpI-containing immunostimulatory oligonucleotide. The invention also provides an immunogenic composition comprising (i) this adjuvant and (ii) an immunogen. The invention also provides a process for preparing an immunogenic composition comprising a step of mixing (i) this adjuvant and (ii) an immunogen. The invention also provides a process for preparing an immunogenic composition of the invention, comprising a step of mixing (i) an oil-in-water emulsion and (ii) an immunogen; and then mixing the emulsion/immunogen mixture with a CpI-containing immunostimulatory oligonucleotide.

The invention also provides a process for preparing an immunogenic composition of the invention, comprising a step of mixing (i) a CpI-containing immunostimulatory oligonucleotide and (ii) an immunogen; and then mixing the oligonucleotide/immunogen mixture with an oil-in-water emulsion.

### The Oil-in-Water Emulsion

Oil-in-water emulsions used with the invention typically include at least one oil and at least one surfactant, with the oil(s) and surfactant(s) being biodegradable (metabolisable) and biocompatible.

The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization. In some useful emulsions at least 80% (by number) of the oil droplets have a diameter less than 500 nm

The emulsions can include oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil, etc. In the grain group, corn oil is the most readily available, but the oil of other cereal grains such as wheat, oats, rye, rice, teff, triticale, etc. may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, are readily available from commercial sources or may be obtained by methods known in the art. Squalene is preferred.

Other useful oils are the tocopherols, which are advantageously included in vaccines for use in elderly subjects (e.g. aged 60 years or older) because vitamin E has been reported to have a positive effect on the immune response in this subject group. They also have antioxidant properties that may help to stabilize emulsions. Various tocopherols exist (α, β, γ, δ, ε or ξ) but α is usually used. A preferred α-tocopherol is DL-α-tocopherol. α-tocopherol succinate is known to be compatible with influenza vaccines and to be a useful preservative as an alternative to mercurial compounds.

Mixtures of oils can be used e.g. squalene and α-tocopherol.

An oil content in the range of 2-20% (by volume) is typical.

Surfactants can be classified by their ‘HLB’ (hydrophile/lipophile balance). Some surfactants useful with the invention have a HLB of at least 10 e.g. at least 15 or at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy(oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. The most preferred surfactant for including in the emulsion is polysorbate 80 (polyoxyethylene sorbitan monooleate; Tween 80).

Mixtures of surfactants can be used e.g. Tween 80/Span 85 mixtures. A combination of a polyoxyethylene sorbitan ester and an octoxynol is also suitable. Another useful combination comprises laureth 9 plus a polyoxyethylene sorbitan ester and/or an octoxynol.

Useful amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, e.g. 0.1 to 10% and in particular 0.1 to 1% or about 0.5%.

Squalene-containing emulsions are preferred, particularly those containing polysorbate 80. Specific oil-in-water emulsion adjuvants useful with the invention include, but are not limited to:
A submicron emulsion of squalene, polysorbate 80, and sorbitan trioleate. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [1-3], as described in more detail in Chapter 10 of ref. 4 and chapter 12 of ref. 5. The MF59 emulsion advantageously includes citrate ions e.g. 10 mM sodium citrate buffer.
A submicron emulsion of squalene, a tocopherol, and polysorbate 80. These emulsions may have from 2 to 10% squalene, from 2 to 10% tocopherol and from 0.3 to 3% polysorbate 80, and the weight ratio of squalene:tocopherol is preferably ≦1 (e.g. 0.90) as this can provide a more stable emulsion. Squalene and polysorbate 80 may be present at a volume ratio of about 5:2 or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion has submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [6].
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL. The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [7] (0.05-1% Thr-MDP, 5% squalane, 2.5% Pluronic L121 and 0.2% polysorbate 80). It can also be used without the Thr-MDP, as in the “AF” adjuvant [8] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [9]. The emulsion may also include one or more of: alditol; a cryoprotective agent (e.g. a sugar, such as dodecylmaltoside and/or sucrose); and/or an alkylpolyglycoside. The emulsion may include a TLR4 agonist [10]. Such emulsions may be lyophilized.
An emulsion of squalene, poloxamer 105 and Abil-Care [11]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 12, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 13, produced by addition of aliphatic amine to desacylsaponin via the carboxyl group of glucuronic acid), dimethyldioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [14].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [15].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [15].

As mentioned above, oil-in-water emulsions comprising squalene are particularly preferred. In some embodiments, the squalene concentration in a vaccine dose may be in the range of 5-15 mg (i.e. a concentration of 10-30 mg/ml, assuming a 0.5 ml dose volume). It is possible, though, to reduce the concentration of squalene [16,17] e.g. to include <5 mg per dose, or even <1.1 mg per dose. For example, a human dose may include 9.75 mg squalene per dose (as in the FLUAD™ product: 9.75 mg squalene, 1.175 mg polysorbate 80, 1.175 mg sorbitan trioleate, in a 0.5 ml dose volume), or it may include a fractional amount thereof e.g. ¾, ⅔, ½, ⅖, ⅓, ¼, ⅕, ⅙, 1/7, ⅛, 1/9, or 1/10. For example, a composition may include 4.875 squalene per dose (and thus 0.588 mg each of polysorbate 80 and sorbitan trioleate), 3.25 mg squalene/dose, 2.438 mg/dose, 1.95 mg/dose, 0.975 mg/dose, etc. Any of these fractional dilutions of the FLUAD™-strength MF59 can be used with the invention, while maintaining a squalene:polysorbate-80:sorbitan-trioleate ratio of 8.3:1:1 (by mass).

### The Immunostimulatory Oligonucleotide and the Polycationic Polymer

The invention uses an immunostimulatory oligonucleotide and a polycationic polymer. These are ideally associated with each other to form a particulate complex, which usefully is a TLR9 agonist.

Immunostimulatory oligonucleotides are known as useful adjuvants. They often contain a CpG motif (a dinucleotide sequence containing an unmethylated cytosine linked to a guanosine) and their adjuvant effect is discussed in refs. 18-23. Oligonucleotides containing TpG motifs, palindromic sequences, multiple consecutive thymidine nucleotides (e.g. TTTT), multiple consecutive cytosine nucleotides (e.g. CCCC) or poly(dG) sequences are also known immunostimulants, as are double-stranded RNAs. Although any of these various immunostimulatory oligonucleotides can be used with the invention, it is preferred to use an oligodeoxynucleotide containing deoxyinosine and/or deoxyuridine, and ideally an oligodeoxynucleotide containing deoxyinosine and deoxycytosine. Inosine-containing oligodeoxynucleotides may include a CpI motif (a dinucleotide sequence containing a cytosine linked to an inosine). The oligodeoxynucleotide may include more than one (e.g. 2, 3, 4, 5, 6 or more) CpI motif, and these may be directly repeated (e.g. comprising the sequence (CI)ₓ, where x is 2, 3, 4, 5, 6 or more) or separated from each other (e.g. comprising the sequence (CIN)ₓ, where x is 2, 3, 4, 5, 6 or more, and where each N independently represents one or more nucleotides). Cytosine residues are ideally unmethylated.

The oligonucleotides will typically have between 10 and 100 nucleotides e.g. 15-50 nucleotides, 20-30 nucleotides, or 25-28 nucleotides. It will typically be single-stranded.

The oligonucleotide can include exclusively natural nucleotides, exclusively non-natural nucleotides, or a mix of both. For instance, it may include one or more phosphorothioate linkage(s), and/or one or more nucleotides may have a 2′-O-methyl modification.

A preferred oligonucleotide for use with the invention is a single-stranded.deoxynucleotide comprising the 26-mer sequence 5′-(IC)₁₃-3′ (SEQ ID NO: 1). This oligodeoxynucleotide forms stable complexes with polycationic polymers to give a good adjuvant.

The polycationic polymer is ideally a polycationic peptide. The polymer may include one or more leucine amino acid residue(s) and/or one or more lysine amino acid residue(s). The polymer may include one or more arginine amino acid residue(s). It may include at least one direct repeat of one of these amino acids e.g. one or more Leu-Leu dipeptide sequence(s), one or more Lys-Lys dipeptide sequence(s), or one or more Arg-Arg dipeptide sequence(s). It may include at least one (and preferably multiple e.g. 2 or 3) Lys-Leu dipeptide sequence(s) and/or at least one (and preferably multiple e.g. 2 or 3) Lys-Leu-Lys tripeptide sequence(s).

The peptide may comprise a sequence R₁—XZXZₓXZX—R₂, wherein: x is 3, 4, 5, 6 or 7; each X is independently a positively-charged natural and/or non-natural amino acid residue; each Z is independently an amino acid residue L, V, I, F or W; and R₁ and R₂ are independently selected from the group consisting of —H, —NH₂, —COCH₃, or —COH. In some embodiments X—R₂ may be an amide, ester or thioester of the peptide's C-terminal amino acid residue.

A polycationic peptide will typically have between 5 and 50 amino acids e.g. 6-20 amino acids, 7-15 amino acids, or 9-12 amino acids.

A peptide can include exclusively natural amino acids, exclusively non-natural amino acids, or a mix of both. It may include L-amino acids and/or D-amino acids. L-amino acids are typical.

A peptide can have a natural N-terminus (NH₂—) or a modified N-terminus e.g. a hydroxyl, acetyl, etc. A peptide can have a natural C-terminus (—COOH) or a modified C-terminus e.g. a hydroxyl, an acetyl, etc. Such modifications can improve the peptide's stability.

A preferred peptide for use with the invention is the 11-mer KLKLLLLLKLK (SEQ ID NO: 2), with all L-amino acids. The N-terminus may be deaminated and the C-terminus may be hydroxylated. A preferred peptide is H—KLKL₅KLK—OH, with all L-amino acids. This oligopeptide is a known antimicrobial [24], neutrophil activator [25] and adjuvant [26] and forms stable complexes with immunostimulatory oligonucleotides to give a good adjuvant.

The most preferred mixture of immunostimulatory oligonucleotide and polycationic polymer is the TLR9 agonist known as IC31™ [27-29], which is an adsorptive complex of oligodeoxynucleotide SEQ ID NO: 1 and polycationic oligopeptide SEQ ID NO: 2.

The oligonucleotide and oligopeptide can be mixed together at various ratios, but they will generally be mixed with the peptide at a molar excess. The molar excess may be at least 5:1 e.g. 10:1, 15:1, 20:1, 25:1, 30:1, 35:1, 40:1 etc. A molar ratio of about 25:1 is ideal [30,31]. Mixing at this excess ratio can result in formation of insoluble particulate complexes between oligonucleotide and oligopeptide. The complexes can be combined with an oil-in-water emulsion.

The oligonucleotide and oligopeptide will typically be mixed under aqueous conditions e.g. a solution of the oligonucleotide can be mixed with a solution of the oligopeptide with a desired ratio. The two solutions may be prepared by dissolving dried (e.g. lyophilised) materials in water or buffer to form stock solutions that can then be mixed.

The complexes can be analysed using the methods disclosed in reference 32. They ideally have an average diameter that is larger than the average diameter of oil droplets in the emulsion. Complexes with an average diameter in the range 1 μm-20 μm can be used. In some embodiments there is no overlap between the size distributions of the emulsion and the complexes i.e. the largest droplets in an emulsion are smaller than the smallest complexes (or the largest complexes are smaller than the smallest droplets). In other embodiments, however, the range of droplet and complex diameters may overlap.

Poly-arginine and CpG oligodeoxynucleotides similarly form complexes [33].

The complexes can be maintained in aqueous suspension e.g. in water or in buffer. Typical buffers for use with the complexes are phosphate buffers (e.g. phosphate-buffered saline), Tris buffers, Tris/sorbitol buffers, borate buffers, succinate buffers, citrate buffers, histidine buffers, etc. As an alternative, complexes may sometimes be lyophilised.

Complexes in aqueous suspension can be centrifuged to separate them from bulk medium (e.g. by aspiration, decanting, etc.). These complexes can then be re-suspended in an alternative medium, such as in an oil-in-water emulsion.

### Mixing of Emulsion, Oligonucleotide and Polymer

Adjuvant compositions of the invention will usually be prepared by mixing an oil-in-water emulsion with an oligonucleotide/polymer complex. The emulsion is a liquid and the complexes are typically maintained in liquid form, and so an adjuvant of the invention may be formed by mixing two liquids.

In some embodiments one or both of the liquids includes an immunogen so that the mixing provides an immunogenic composition of the invention. In other embodiments neither liquid includes an immunogen, so the mixed product (i.e. the adjuvant composition of the invention) can later be combined with an immunogen to provide an immunogenic composition of the invention.

Where two liquids are mixed the volume ratio for mixing can vary (e.g. between 20:1 and 1:20, between 10:1 and 1:10, between 5:1 and 1:5, between 2:1 and 1:2, etc.) but is ideally about 1:1. The concentration of components in the two liquids can be selected so that a desired final concentration is achieved after mixing e.g. both may be prepared at 2× strength such that 1:1 mixing provides the final desired concentrations.

In other embodiments the complexes are not in liquid form (e.g. they have been centrifuged or lyophilised) and they may be combined with (e.g. dissolved in) an emulsion.

Various concentrations of oligonucleotide and polycationic polymer can be used e.g. any of the concentrations used in references 27, 30 or 31, or in reference 34. For example, a polycationic oligopeptide can be present at 1100 μM, 1000 μM, 350 μM, 220 μM, 200 μM, 110 μM, 100 μM, 11 μM, 10 μM, 500 nM, 50 nM, etc. An oligonucleotide can be present at 44 nM, 40 nM, 20 nM, 14 nM, 4.4 nM, 4 nM, etc. A polycationic oligopeptide concentration of less than 2000 nM is typical. For SEQ ID NOs: 1 & 2, mixed at a molar ratio of 1:25, the concentrations in mg/mL in three embodiments of the invention may thus be 0.311 & 1.322, or 0.109 & 0.463, or 0.031 and 0.132.

Mixing a squalene-in-water emulsion with an aqueous preparation of IC31 at a 1:1 volume ratio can be used to give a composition with the final amounts of components per ml: squalene, 4.9 mg; polysorbate 80, 588 μg; cationic oligopeptide, 500 nMol or 50 nMol; oligonucleotide, 20 nMol or 2 nMol.

### Pharmaceutical Compositions

Adjuvant compositions of the invention usually include components in addition to the emulsion, oligonucleotide and polymer e.g. they typically include one or more pharmaceutically acceptable component. Such components may also be present in immunogenic compositions of the invention, originating either in the adjuvant composition or in another composition. A thorough discussion of such components is available in reference 35.

A composition may include a preservative such as thiomersal or 2-phenoxyethanol. It is preferred that the vaccine should be substantially free from (e.g. <10 μg/ml) mercurial material e.g. thiomersal-free. Vaccines containing no mercury are more preferred. Preservative-free vaccines are particularly preferred. α-tocopherol succinate can be included as an alternative to mercurial compounds in influenza vaccines.

To control tonicity, a composition may include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate, and/or magnesium chloride, etc.

Compositions may have an osmolality of between 200 mOsm/kg and 400 mOsm/kg, e.g. between 240-360 mOsm/kg, maybe within the range of 280-330 mOsm/kg or 290-310 mOsm/kg.

The pH of a composition will generally be between 5.0 and 8.1, and more typically between 6.0 and 8.0 e.g. 6.5 and 7.5, or between 7.0 and 7.8.

A composition is preferably sterile. A composition is preferably non-pyrogenic e.g. containing <1 EU (endotoxin unit, a standard measure) per dose, and preferably <0.1 EU per dose. A composition is preferably gluten free.

An immunogenic composition may include material for a single immunisation, or may include material for multiple immunisations (i.e. a ‘multidose’ kit). The inclusion of a preservative is useful in multidose arrangements. As an alternative (or in addition) to including a preservative in multidose compositions, the compositions may be contained in a container having an aseptic adaptor for removal of material.

Compositions will generally be in aqueous form at the point of administration. Vaccines are typically administered in a dosage volume of about 0.5 ml, although a half dose (i.e. about 0.25 ml) may sometimes be administered e.g. to children. In some embodiments of the invention a composition may be administered in a higher dose e.g. about 1 ml e.g. after mixing two 0.5 ml volumes.

### Immunogens

Adjuvant compositions of the invention can be administered to animals in combination with immunogens to induce an immune response. The invention can be used with a wide range of immunogens, for treating or protecting against a wide range of diseases. The immunogen may elicit an immune response that protects against a viral disease (e.g. due to an enveloped or non-enveloped virus), a bacterial disease (e.g. due to a Gram negative or a Gram positive bacterium), a fungal disease, a parasitic disease, an auto-immune disease, or any other disease. The immunogen may also be useful in immunotherapy e.g. for treating a tumour/cancer, Alzheimer's disease, or an addiction.

The immunogen may take various forms e.g. a whole organism, an outer-membrane vesicle, a protein, a saccharide, a liposaccharide, a conjugate (e.g. of a carrier and a hapten, or of a carrier and a saccharide or liposaccharide), etc.

The immunogen may elicit an immune response against an influenza virus, including influenza A and B viruses. The presence of an oil-in-water emulsion adjuvant (particularly one comprising squalene) has been shown to enhance the strain cross-reactivity of immune responses for seasonal [36] and pandemic [37,38] influenza vaccines. Various forms of influenza virus immunogen are currently available, typically based either on live virus or on inactivated virus. Inactivated vaccines may be based on whole virions, split virions, or on purified surface antigens. Influenza antigens can also be presented in the form of virosomes. Hemagglutinin is the main immunogen in current inactivated vaccines, and vaccine doses are standardised by reference to HA levels, typically measured by SRID. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used, as have higher doses (e.g. 3× or 9× doses [39,40]). Thus compositions may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 45, about 30, about 15, about 10, about 7.5, about 5, about 3.8, about 1.9, about 1.5, etc. per strain. It is usual to include substantially the same mass of HA for each strain included in the vaccine e.g. such that the HA mass for each strain is within 10% of the mean HA mass per strain, and preferably within 5% of the mean. For live vaccines, dosing is measured by median tissue culture infectious dose (TCID₅₀) rather than HA content, and a TCID₅₀ of between 10⁶ and 10⁸ (preferably between 10^{6.5}-10^{7.5}) per strain is typical. Rather than use SPF eggs as the substrate for viral growth, where virus is harvested from infected allantoic fluids of hens' eggs, cell lines that support influenza virus replication may be used. The cell line will typically be of mammalian origin e.g. MDCK. Influenza A virus immunogens may be from any suitable HA subtype strain e.g. H1, H3, H5, H7, H9 etc., such as a H1N1, H3N2 and/or H5N1 strain.

The immunogen may elicit an immune response against a Candida fungus such as C. albicans. For instance, the immunogen may be a β-glucan, which may be conjugated to a carrier protein. The glucan may include β-1,3 and/or β-1,6 linkages. Suitable immunogens include those disclosed in references 41 & 42.

The immunogen may elicit an immune response against a Streptococcus bacterium, including S. agalactiae, S. pneumoniae and S. pyogenes. For instance, the immunogen may be a capsular saccharide, which may be conjugated to a carrier protein. For S. agalactiae the saccharide may be from one or more of serotypes Ia, Ib, II, III, and/or V. For S. pneumoniae the saccharide may be from one or more of serotypes 1, 3, 4, 5, 6B, 7F, 9V, 14, 18C, 19F, and/or 23F. In addition to (or in place of) capsular saccharide immunogen(s), polypeptide immunogens may be used to elicit a protective anti-streptococcal immune response.

The immunogen may elicit an immune response against a meningococcal bacterium (N. meningitidis). For instance, the immunogen may be a capsular saccharide, which may be conjugated to a carrier protein. Capsular saccharides and their conjugates are particularly useful for protecting against meningococcal serogroups A, C, W135 and/or Y. In addition to (or in place of) capsular saccharide immunogen(s), polypeptide immunogens and/or outer membrane vesicles may be used to elicit a protective anti-meningococcal immune response, particularly for use against serogroup B e.g. as disclosed in reference 43.

The immunogen may elicit an immune response against a hepatitis virus, such as a hepatitis A virus, a hepatitis B virus and/or a hepatitis C virus. For instance, the immunogen may be hepatitis B virus surface antigen (HBsAg).

The immunogen may elicit an immune response against a respiratory syncytial virus. Immunogens may be from a group A RSV and/or a group B RSV. Suitable immunogens may comprise the F and/or G glycoproteins or fragments thereof e.g. as disclosed in references 44 & 45.

The immunogen may elicit an immune response against a Chlamydia bacterium, including C. trachomatis and C. pneumoniae. Suitable immunogens include those disclosed in references 46-52.

The immunogen may elicit an immune response against an Escherichia coli bacterium, including extraintestinal pathogenic strains. Suitable immunogens include those disclosed in references 53-55

The immunogen may elicit an immune response against a coronavirus, such as the human SARS coronavirus. Suitable immunogens may comprise the spike glycoprotein.

The immunogen may elicit an immune response against a Helicobacter pylori bacterium. Suitable immunogens include CagA [56-59], VacA [60,61], and/or NAP [62-64].

The immunogen may elicit an immune response against rabies virus. A suitable immunogen is an inactivated rabies virus [65, RabAvert™]

The immunogen may elicit an immune response against a human papillomavirus. Useful immunogens are L1 capsid proteins, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L1 gene(s). More preferably, the composition includes L1 VLPs from both HPV-16 and HPV-18 strains. This bivalent combination has been shown to be highly effective [66]. In addition to HPV-16 and HPV-18 strains, it is also possible to include L1 VLPs from HPV-6 and HPV-11 strains.

The immunogen may elicit an immune response against a tumour antigen, such as MAGE-1, MAGE-2, MAGE-3 (MAGE-A3), MART-1/Melan A, tyrosinase, gp100, TRP-2, etc. The immunogen may elicit an immunotherapeutic response against lung cancer, melanoma, breast cancer, prostate cancer, etc.

The immunogen may elicit an immune response against a hapten conjugated to a carrier protein, where the hapten is a drug of abuse [67]. Examples include, but are not limited to, opiates, marijuana, amphetamines, cocaine, barbituates, glutethimide, methyprylon, chloral hydrate, methaqualone, benzodiazepines, LSD, nicotine, anticholinergic drugs, antipsychotic drugs, tryptamine, other psychomimetic drugs, sedatives, phencyclidine, psilocybine, volatile nitrite, and other drugs inducing physical and/or psychological dependence.

Various other immunogens may be used.

Where an immunogenic composition includes a complex of an immunostimulatory oligonucleotide and a polycationic polymer, immunogens will usually be adsorbed to the complexes, but this is not required. Thus antigens may, after centrifugation, be associated with the complexes, indicating adsorption. Where an immunogen is described as being “at least partially adsorbed” to a complex, it is preferred that at least 10% (by weight) of the total amount of that immunogen in the composition is adsorbed e.g. >20%, >30%, >40% or more. Where an immunogen is described as being “adsorbed” to a complex, it is preferred that at least 50% (by weight) of the total amount of that immunogen in the composition is adsorbed e.g. 50%, 60%, 70%, 80%, 90%, 95%, 98% or more. In some embodiments an immunogen is totally adsorbed i.e. none is detectable in the supernatant after centrifugation to separate complexes from bulk liquid medium. In other embodiments, though, there is no adsorption.

### Packaging of Compositions or Kit Components

Suitable containers for adjuvant compositions, immunogenic compositions and kit components of the invention include vials, syringes (e.g. disposable syringes), etc. These containers should be sterile. The containers can be packaged together to form a kit e.g. in the same box.

Where a component is located in a vial, the vial can be made of a glass or plastic material. The vial is preferably sterilized before the composition is added to it. To avoid problems with latex-sensitive subjects, vials are preferably sealed with a latex-free stopper, and the absence of latex in all packaging material is preferred. The vial may include a single dose of vaccine, or it may include more than one dose (a ‘multidose’ vial) e.g. 10 doses. Useful vials are made of colorless glass. Borosilicate glasses are preferred to soda lime glasses. Vials may have stoppers made of butyl rubber.

A vial can have a cap (e.g. a Luer lock) adapted such that a syringe can be inserted into the cap. A vial cap may be located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached, a separate needle may be supplied with the syringe for assembly and use. Such a needle may be sheathed. The plunger in a syringe may have a stopper to prevent the plunger from being accidentally removed during aspiration. The syringe may have a latex rubber cap and/or plunger. Disposable syringes contain a single dose of vaccine. The syringe will generally have a tip cap to seal the tip prior to attachment of a needle, and the tip cap may be made of a butyl rubber. If the syringe and needle are packaged separately then the needle is preferably fitted with a butyl rubber shield. Useful syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

It is usual in multi-component products to include more material than is needed for subject administration, so that a full final dose volume is obtained despite any inefficiency in material transfer. Thus an individual container may include overfill e.g. of 5-20% by volume.

### Methods of Treatment, and Administration of Immunogenic Compositions

Compositions of the invention are suitable for administration to human subjects, and the invention provides a method of raising an immune response in a subject, comprising the step of administering an immunogenic composition of the invention to the subject.

The invention also provides a method of raising an immune response in a subject, comprising the step of mixing the contents of the containers of a kit of the invention (or chambers of a syringe) and administering the mixed contents to the subject.

The invention also provides composition or kit of the invention for use as a medicament e.g. for use in raising an immune response in a subject.

The invention also provides the use of an oil-in-water emulsion, an immunostimulatory oligonucleotide and a polycationic polymer, in the manufacture of a medicament for raising an immune response in a subject. This medicament may be administered in combination with an immunogen.

The invention also provides the use of an oil-in-water emulsion, an immunostimulatory oligonucleotide, a polycationic polymer and an immunogen, in the manufacture of a medicament for raising an immune response in a subject.

These methods and uses will generally be used to generate an antibody response, preferably a protective antibody response.

Compositions of the invention can be administered in various ways. The usual immunisation route is by intramuscular injection (e.g. into the arm or leg), but other available routes include subcutaneous injection, intranasal, oral, buccal, sublingual, intradermal, transcutaneous, transdermal, etc.

Immunogenic compositions prepared according to the invention may be used as vaccines to treat both children and adults. A subject may be less than 1 year old, 1-5 years old, 5-15 years old, 15-55 years old, or at least 55 years old. Preferred subjects for receiving the vaccines are the elderly (e.g. ≧50 years old, ≧60 years old, and preferably ≧65 years), the young (e.g. ≧5 years old), hospitalised subjects, healthcare workers, armed service and military personnel, pregnant women, the chronically ill, immunodeficient subjects, people travelling abroad, etc. IC31™ has been shown to be effective in infant populations [34, 68]. The vaccines are not suitable solely for these groups, however, and may be used more generally in a population.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve subjects. Multiple doses will typically be administered at least 1 week apart (e.g. about 2 weeks, about 3 weeks, about 4 weeks, about 6 weeks, about 8 weeks, about 12 weeks, about 16 weeks, etc.).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, or for viral growth, it is preferably one that has been approved for use in human vaccine production e.g. as in Ph Eur general chapter 5.2.3.

### BRIEF DESCRIPTION OF DRAWINGS

FIG. 1 shows the particle diameters in a mixture of IC31 and MF59. The y-axis shows volume (%) and the x-axis shows particle diameter (μm).

FIGS. 2 to 5 show the same analysis for adjuvanted H5N1 antigen at: (2) time zero; (3) 30 minutes; (4) 6 hours; and (5) 24 hours.

FIG. 6 shows the same analysis for H5N1 antigen adjuvanted with IC31 alone.

FIGS. 7 and 8 show body temperature (° C.) over time, from 3 days before infection to 5 days after.

### MODES FOR CARRYING OUT THE INVENTION

### Adjuvants

A squalene-in-water emulsion, MF59, was prepared as disclosed in Chapter 10 of reference 4. IC31 was prepared in high and low concentrations (10-fold difference) as disclosed in reference 31. Adjuvant combinations were made my mixing MF59 with IC31^{high} or IC31^{low} at either a 1:1 volume ratio or a 5:1 volume ratio. The three individual adjuvants (MF59, IC31^{high}, IC31^{low}), and the two mixtures (MF59+IC31^{high}, MF59+IC31^{low}), have been combined with various immunogens and administered to a variety of mammals to assess their efficacy.

In addition, IC31^{high} and IC31^{low} have been mixed with a MF59-adjuvanted influenza vaccine (FLUAD™) at a 1:1 volume ratio. For human subjects the mixing is performed immediately prior to immunisation (“bedside mix”).

### Influenza Virus

After addition of either IC31^{high} or IC31^{low} to FLUAD™ the influenza antigens rapidly associate with (adsorb to) the IC31 complexes. At room temperature, within 30 minutes of adding IC31^{high} at least 98% of the antigens adsorb to the IC31 particles. 96% adsorption was seen 2 hours after adding IC31^{low} at room temperature. At a lower temperature (4° C.) for 24 hours, 97% (IC31^{high}) or 91% (IC31^{low}) adsorption was seen. Osmolality and pH remained substantially constant over 24 hours at room temperature for both IC31^{high} and IC31^{low}, indicating that the combinations are stable. The particle sizes of the emulsion and of the IC31 complexes are maintained after mixing.

Influenza antigens were adjuvanted either with MF59 (FLUAD™), with IC31 (either IC31^{high} or IC31^{low}) or with a combination of MF59+IC31 (50 μL FLUAD™ mixed with equal volume of aqueous IC31; both adjuvants mixed at 2× strength to give final 1× after dilution). Two doses of the various compositions were administered to mice and HI titres were assessed. Based on single samples, titres after the second dose against a H1N1 strain of influenza A virus were:

| Antigen | − | + | + | + | + | + | + |
|---|---|---|---|---|---|---|---|
| Adjuvant | − | − | MF59 | IC31^{hi} | IC31^{lo} | MF59 + | MF59 + |
| | | | | | | IC31^{hi} | IC31^{lo} |
| Titre | 84 | 400 | 3360 | 1392 | 704 | 3200 | 4352 |

Looking at pooled samples for all three strains, HI titres were as follows

| Antigen | − | + | + | + | + | + | + |
|---|---|---|---|---|---|---|---|
| Adjuvant | − | − | MF59 | IC31^{hi} | IC31^{lo} | MF59 + | MF59 + |
| | | | | | | IC31^{hi} | IC31^{lo} |
| H1N1 titre | 160 | 640 | 3840 | 1920 | 640 | 10240 | 10240 |
| H3N2 titre | 240 | 1920 | 5120 | 5120 | 640 | 7880 | 7880 |
| B titre | 20 | 320 | 1920 | 640 | 480 | 640 | 1280 |

Thus the combination of MF59 and IC31 can enhance HI titres more than either adjuvant alone, particularly for influenza A virus.

CD4+ T cells were assessed to determine whether the adjuvants elicited a Th1-type or Th2-type response. Whereas MF59 gave a response that was biased towards a Th2-type response, and IC31 gave a response (at both doses) that was biased towards a Th1-type response (but less strongly biased than MF59), the combined adjuvant was more balanced between Th1-type and Th2-type responses.

Although addition of IC31 to FLUAD™ does not have a large impact on HI titres, it does shift the balance of the immune response.

### Hepatitis C Virus

Hepatitis C virus E1E2 protein was used to immunise mice. The antibody response achieved using MF59+IC31 was similar to the response with MF59 alone, but the highest inhibition of CD81 binding after 3 doses was seen with MF59+IC31^{low}.

### ExPEC

Antigen 124 from an extraintestinal pathogenic E. coli strain was adjuvanted with alum, IC31, MF59 or IC31+MF59. Protection rates were as follows:

| Alum | IC31 | MF59 | MF59 + IC31 |
|---|---|---|---|
| 40% | 66% | 75% | 83% |

### Serogroup B Meningococcus

The antigens from the meningococcus serogroup B vaccine of reference 43 were adjuvanted with MF59, IC31^{high}, IC31^{low} or combinations thereof. The vaccine includes three recombinant antigens (Ag1, Ag2 & Ag3) and total IgG levels against these were as follows:

| | Antigen | | |
|---|---|---|---|
| | Ag1 | Ag2 | Ag3 |
| IC31^{low} | 7025 | 2021 | 2357 |
| MF59 + IC31^{low} | 27132 | 7448 | 7120 |
| MF59 | 28611 | 4436 | 13099 |
| MF59 + IC31^{high} | 36541 | 13632 | 8560 |
| IC31^{high} | 21334 | 6922 | 12962 |

Except for antigen ‘Ag3’, therefore, the highest IgG levels were seen when using a mixture of MF59 and IC31.

Sera were also tested for their bactericidal activity against various meningococcal strains. Representative results include:

| | Strain | | | | | | | |
|---|---|---|---|---|---|---|---|---|
| | A | B | C | D | E | F | G | H |
| IC31^{low} | 1024 | 256 | 4096 | 2048 | 256 | 64 | 512 | <16 |
| MF59 + | 4096 | 1024 | 4096 | 2048 | 1024 | 128 | 4096 | <16 |
| IC31^{low} | | | | | | | | |
| MF59 | 32768 | 1024 | 32768 | 4096 | 2048 | 128 | 4096 | <16 |
| MF59 + | 8192 | 2048 | 8192 | 32768 | 2048 | 128 | 8192 | <16 |
| IC31^{high} | | | | | | | | |
| IC31^{high} | 16384 | 2048 | 16384 | 32768 | 2048 | 512 | 4096 | <16 |

With some exceptions, therefore, the highest bactericidal titres were seen when using a mixture of MF59 and IC31.

### Multiple Serogroups of Meningococcal

These meningococcal B protein antigens were also combined with conjugated saccharide antigens from serogroups A, C, W135 and Y antigens and were tested with the same adjuvant mixtures. Bactericidal titres against a test strain from each serogroup were as follows:

Antigen

| | A | C | W135 | Y |
|---|---|---|---|---|
| IC31^{low} | 16384 | 8192 | 1024 | 2048 |
| MF59 + IC31^{low} | 4096 | 8192 | 4096 | 8192 |
| MF59 | 16384 | 8192 | 2048 | 4096 |
| MF59 + IC31^{high} | 8192 | 16384 | 4096 | 4096 |
| IC31^{high} | 32768 | 16384 | 4096 | 4096 |

Except for serogroup A, therefore, the highest bactericidal titres were seen when using a mixture of MF59 and IC31.

### Pandemic Influenza

IC31^{high} was combined with MF59 or with buffer at a 1:1 ratio, or with MF59 and buffer at a 1:0.5:0.5 ratio. Thus MF59 was tested at its normal concentration or at half concentration. The adjuvants were combined with a surface antigen vaccine from a H5N1 strain of influenza virus (A/Vietnam/1193/04). The stability and immunogenicity of the combination was tested.

Stability was evaluated by testing pH, osmolality, adsorption, and particle size at time zero and then after 30 minutes or 6 hours of storage at room temperature. The pH was stable in the range 7.23 to 7.26. Osmolality was stable in the range 280-286 mOsm/kg. The proportion of adsorbed antigen rose from 76% to 80% over 6 hours, having dropped slightly at the 30 minute time point. These figures are similar to control compositions lacking MF59, which had slightly lower osmolality (273-275 mOsm/kg), a slightly higher pH (7.32-7.35), and a slightly lower proportion of adsorbed antigen (54-68%).

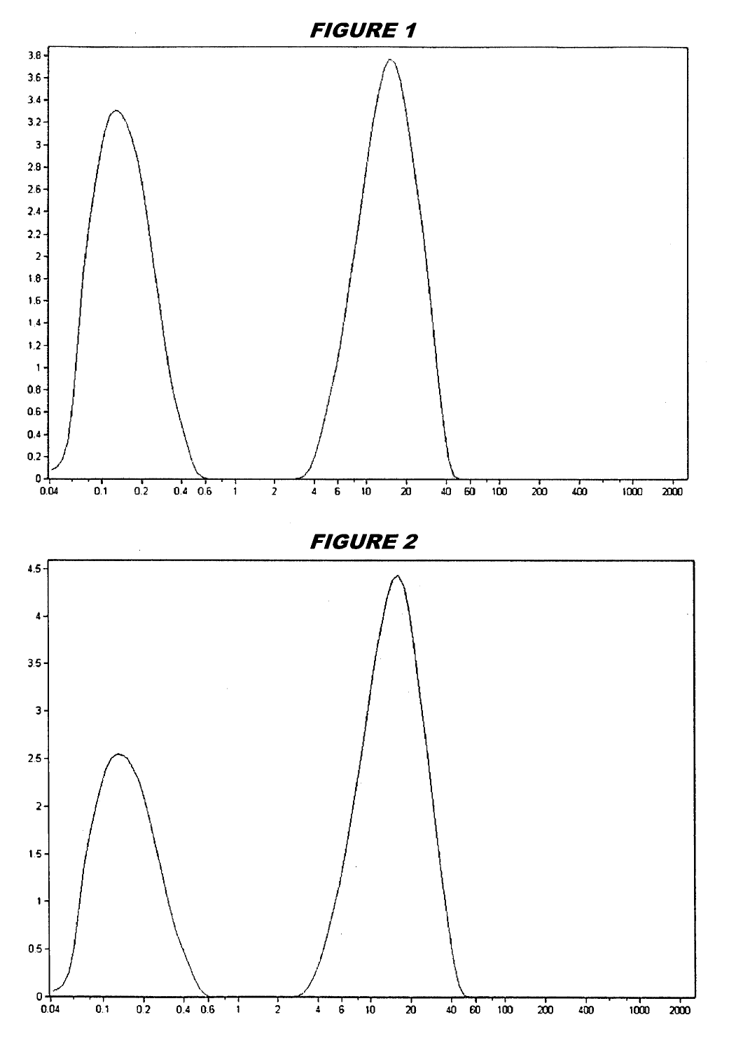

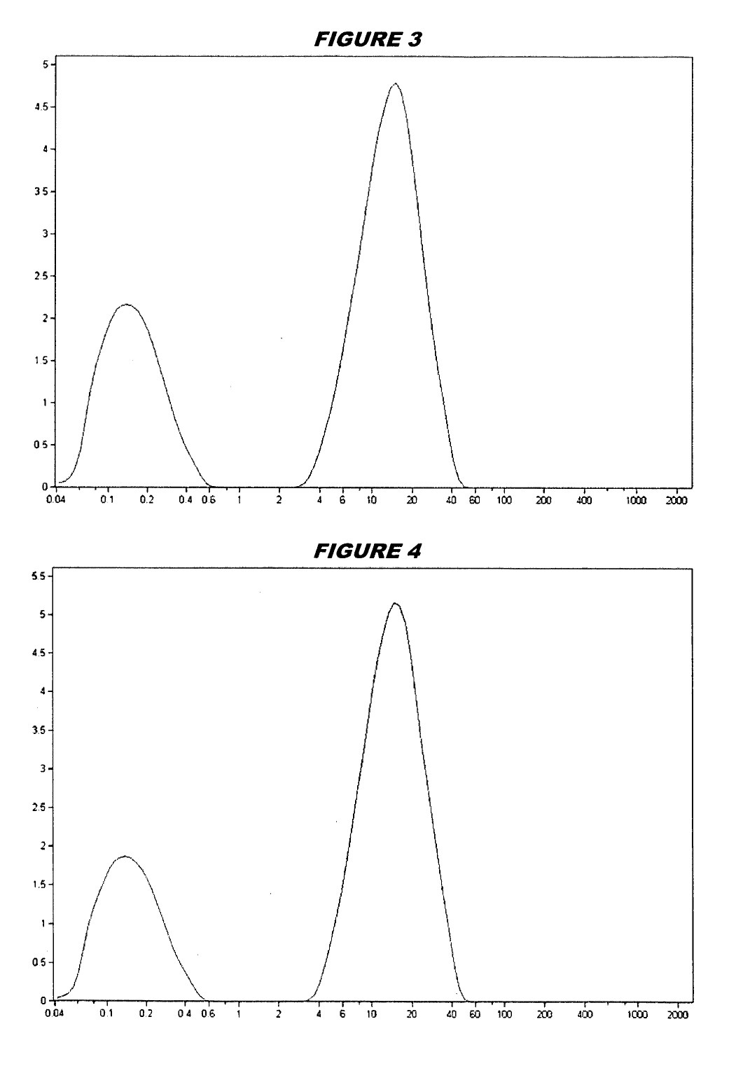

The particle sizes of a IC31^{high}:MF59 1:1 mixture in PBS, at time zero, are shown in FIG. 1. The emulsion droplets (mean diameter 161 nm) accounted for 46.4% of the volume and IC31 complexes (mean diameter 15.9 μm) for 53.6%. FIGS. 2 to 5 show similar analysis but for an adjuvanted vaccine including H5N1 antigen, from time zero through to 24 hours at room temperature:

| | MF59 | | IC31 | |
|---|---|---|---|---|
| | | Mean diameter | | Mean diameter |
| | Volume % | (nm) | Volume % | (μm) |
| (2) Time 0 | 37.1 | 166 | 62.9 | 16.2 |
| (3) 30 minutes | 32.3 | 171 | 67.7 | 15.1 |
| (4) 6 hours | 27.5 | 169 | 72.5 | 16.1 |
| (5) 24 hours | 25.0 | 173 | 75.0 | 16.4 |

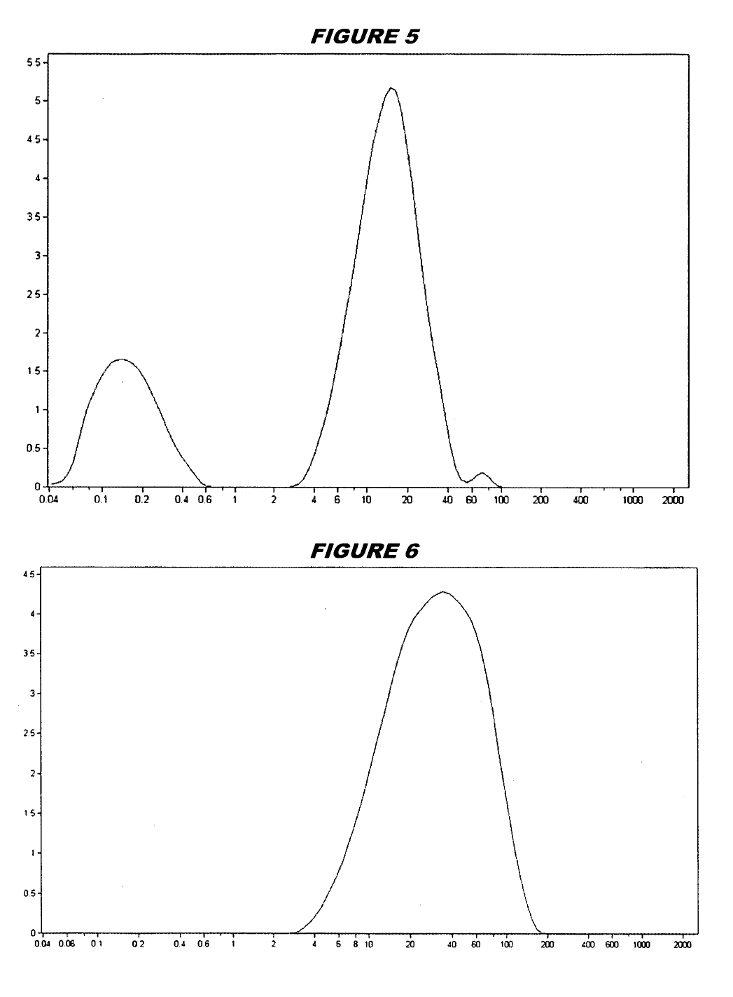

H5N1 antigen in combination with MF59 alone (no IC31) had a mean droplet diameter of 151 nm. H5N1 antigen in combination with IC31^{high} (but no MF59) had a mean particle diameter of 38.4 μm (FIG. 6). Thus the mixing of MF59 and IC31 slightly increases the analysed diameter of MF59 particles (while still permitting sterile filtration) and reduces the diameter of the IC31 complexes. In all cases, though, the mixtures are stable.

The ferret (Mustela putorius faro) model is the preferred animal model to provide evidence of efficacy of candidate pandemic influenza vaccines. Thus adjuvanted vaccines with either 1 μg of 3.75 μg of antigen (hemagglutinin dose) were administered to eight groups of ferrets. Ferrets received a priming dose and a boosting dose, and were then challenged with a heterologous H5N1 strain.

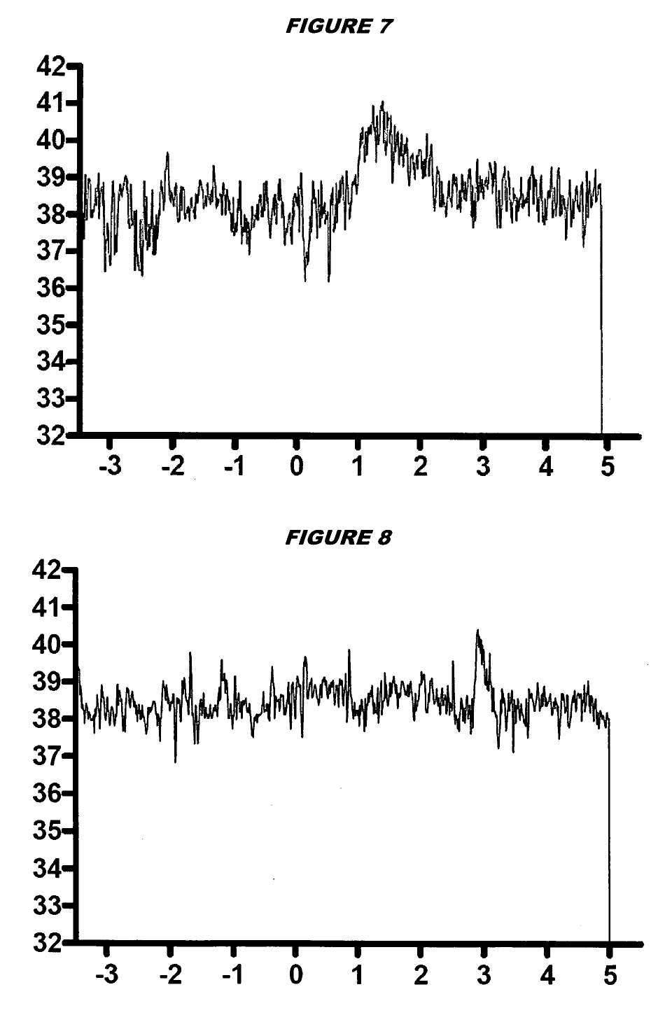

Body temperature of ferrets was monitored before and after challenge. FIGS. 7 and 8 show temperatures from two example mice, one in group D (FIG. 7) and one in group E (FIG. 8).

HAI titers were assessed against the vaccine strain on days 0, 21, 42 and 49. Average titers were:

| | Day 0 | Day 21 | Day 42 | Day 49 |
|---|---|---|---|---|
| A | 5 | 5 | 28 | 24 |
| B | 5 | 31 | 313 | 265 |
| C | 5 | 5 | 38 | 29 |
| D | 5 | 47 | 293 | 182 |
| E | 5 | 60 | 820 | 578 |
| F | 5 | 5 | 5 | 5 |
| G | 5 | 5 | 28 | 20 |
| H | 5 | 5 | 5 | 5 |

Thus the combination of IC31 and MF59 (groups B and E) gave the highest titers.

Preliminary data looked at the percentage of affected lung tissue (estimation of the area of macroscopic lung lesions) and relative lung weights (below 1.0 indicates for a healthy lung). Mean results per group were as follows:

| | | | | Relative |
|---|---|---|---|---|
| | | | | lung |
| | Prime | Boost | % affected | weight |
| A | 1 μg + MF59 | 1 μg + MF59 | 25.8 | 1.1 |
| B | 1 μg + IC31/MF59 | 1 μg + IC31/MF59 | 0.0 | 0.9 |
| C | 3.75 μg + MF59 | 3.75 μg + MF59 | 18.3 | 1.3 |
| D | 3.75 μg + IC31 | 3.75 μg + IC31 | 2.5 | 1.0 |
| E | 3.75 μg + IC31/MF59 | 3.75 μg + IC31/MF59 | 1.7 | 1.0 |
| F | — | 3.75 μg + MF59 | 16.7 | 1.9 |
| G | — | 3.75 μg + IC31/MF59 | 14.0 | 1.1 |
| H | IC31/MF59 | IC31/MF59 | 87.1 | 2.1 |

The combination of IC31 and MF59 improved the lung pathology in this model when compared to MF59 or IC31 alone.

Thus the combination of IC31 and with the MF59 oil-in-water emulsion adjuvant was stable and provided good immunogenicity and protection against a pandemic influenza virus strain to ferrets.

### Group A Streptococcus and Candida

S. pyogenes and C. albicans antigens have also been adjuvanted with MF59™+IC31™.

It will be understood that the invention has been described by way of example only and modifications may be made whilst remaining within the scope and spirit of the invention.

### REFERENCES

[1] WO90/14837.
[2] Podda & Del Giudice (2003) Expert Rev Vaccines 2:197-203.
[3] Podda (2001) Vaccine 19: 2673-2680.
[4] Vaccine Design: The Subunit and Adjuvant Approach (eds. Powell & Newman) Plenum Press 1995 (ISBN 0-306-44867-X).
[5] Vaccine Adjuvants: Preparation Methods and Research Protocols (Volume 42 of Methods in Molecular Medicine series). ISBN: 1-59259-083-7. Ed. O'Hagan.
[6] WO2008/043774.
[7] Allison & Byars (1992) Res Immunol 143:519-25.
[8] Hariharan et al. (1995) Cancer Res 55:3486-9.
[9] US-2007/0014805.
[10] US-2007/0191314.
[11] Suli et al. (2004) Vaccine 22 (25-26):3464-9.
[12] WO95/11700.
[13] U.S. Pat. No. 6,080,725.
[14] WO2005/097181.
[15] WO2006/113373.
[16] WO2007/052155.
[17] WO2008/128939.
[18] Krieg (2003) Nature Medicine 9:831-835.
[19] McCluskie et al. (2002) FEMS Immunology and Medical Microbiology 32:179-185.
[20] WO98/40100.
[21] U.S. Pat. No. 6,207,646.
[22] U.S. Pat. No. 6,239,116.
[23] U.S. Pat. No. 6,429,199.
[24] Alvarez-Bravo et al. (1994) Biochem J 302:535-8.
[25] Nakajima et al. (1997) FEBS Letts 415:64-66.
[26] Fritz et al. (2004) Vaccine 22:3274-84.
[27] Schellack et al. (2006) Vaccine 24:5461-72.
[28] Lingnau et al. (2007) Expert Rev Vaccines 6:741-6.
[29] WO2004/084938.
[30] Kamath et al. (2008) Eur J Immunol 38:1247-56.
[31] Riedl et al. (2008) Vaccine 26:3461-8.
[32] Kritsch et al. (2005) J Chromatography B 822:263-70.
[33] Lingnau et al. (2003) Vaccine 20:3498-508.
[34] Olafsdottir et al. (2009) Scand J Immunol 69:194-202.
[35] Gennaro (2000) Remington: The Science and Practice of Pharmacy. 20th edition, ISBN: 0683306472.
[36] O'Hagan (2007) Expert Rev Vaccines. 6 (5):699-710.
[37] Bernstein et al. (2008)J Infect Dis. 197 (5):667-75.
[38] Stephenson et al. (2005) J Infect Dis. 191 (8):1210-5.
[39] Treanor et al. (1996) J Infect Dis 173:1467-70.
[40] Keitel et al. (1996) Clin Diagn Lab Immunol 3:507-10.
[41] WO03/097091.
[42] Cassone & Torosantucci (2006) Expert Rev Vaccines 5:859-67.
[43] Giuliani et al. (2006) Proc Natl Acad Sci USA. 103:10834-9.
[44] WO95/27787.
[45] WO03/010317.
[46] WO2007/110700.
[47] WO2006/138004.
[48] WO2005/084306.
[49] WO2005/002619.
[50] WO03/049762.
[51] WO02/02606.
[52] WO00/37494.
[53] WO2008/020330.
[54] WO2006/091517.
[55] WO2006/089264.
[56] Covacci & Rappuoli (2000) J. Exp. Med. 19:587-592.
[57] WO 93/18150.
[58] Covacci et al. (1993) Proc. Natl. Acad. Sci. USA 90:5791-5795.
[59] Tummuru et al. (1994) Infect. Immun. 61:1799-1809.
[60] Marchetti et al. (1998) Vaccine 16:33-37.
[61] Telford et al. (1994) J. Exp. Med. 179:1653-1658.
[62] Evans et al. (1995) Gene 153:123-127.
[63] WO 96/01272 & WO96/01273, especially SEQ ID NO:6.
[64] WO 97/25429.
[65] MMWR Morb Mortal Wkly Rep 1998 Jan. 16; 47 (1):12, 19.
[66] Harper et al. (2004) Lancet 364 (9447):1757-65.
[67] U.S. Pat. No. 6,699,474.
[68] Kamath et al. (2008) PLoS ONE 3 (11):e3683.

1. An immunological adjuvant comprising an oil-in-water emulsion, an immunostimulatory oligonucleotide and a polycationic polymer.

2. The adjuvant of claim 1, wherein the oligonucleotide and the polymer are complexed.

3. The adjuvant of claim 2, wherein the complexes of oligonucleotide and polymer are adsorptive.

4. The adjuvant of claim 3, wherein the average diameter of the adsorptive complexes is greater than the average diameter of oil droplets in the emulsion.

5. The adjuvant of claim 4, wherein the complexes have an average diameter in the range 1-20 μm.

6. The adjuvant of claim 5, wherein the oil droplets have an average diameter of <220 nm.

7. The adjuvant of claim 4, wherein there is no overlap between the size distributions of the emulsion and the complexes.

8. An immunological adjuvant comprising an oil-in-water emulsion and an adsorptive particulate adjuvant, wherein the average diameter of particles in the adsorptive particulate adjuvant is greater than the average diameter of oil droplets in the emulsion, and wherein the adsorptive particulate adjuvant is a complex of an immunostimulatory oligonucleotide and a polycationic polymer.

9. An immunological adjuvant comprising an oil-in-water emulsion and an immunostimulatory oligonucleotide, wherein the immunostimulatory oligonucleotide includes at least one CpI motif.

10. An immunological adjuvant comprising an oil-in-water emulsion and an adsorptive particulate adjuvant, wherein the average diameter of particles in the adsorptive particulate adjuvant and the average diameter of oil droplets in the emulsion are both less than 250 nm.

11. A process for preparing the adjuvant of claim 1, comprising a step of mixing an oil-in-water emulsion with a complex of an immunostimulatory oligonucleotide and a polycationic polymer.

12. An immunogenic composition comprising (i) the adjuvant of claim 1 and (ii) an immunogen.

13. A process for preparing an immunogenic composition comprising a step of mixing (i) the adjuvant of claim 1 and (ii) an immunogen.

14. The composition of claim 12, wherein the immunogen, when administered to a host, elicits an immune response that protects against a viral disease, a bacterial disease, a fungal disease, a parasitic disease, or an auto-immune disease.

15. The composition of claim 12, wherein the immunogen elicits an immune response against an influenza A or B virus e.g. a H5N1 influenza A virus.
